(19) United States
(12) Patent Application Publication
(10) Pub. No.: US 2010/0204313 A1
(43) Pub. Date: Aug. 12, 2010

(54) Modulation of nerve pain activity by resiniferatoxin and uses thereof

(76) Inventor: Pankaj J. Pasricha, Cupertino, CA (US)

Correspondence Address: Benjamin Aaron Adler;ADLER & ASSOCIATES, 8011 Candle Lane, Houston, TX, 77071, US

(21) Appl. No.: 12/799,039

(22) Filed: Apr. 16, 2010

Related U.S. Application Data
(63) Continuation of application No. PCT/US2008/011890, filed on Oct. 17, 2008.
(60) Provisional application No. 60/988,409, filed on Nov. 15, 2007.

(51) Int. Cl. A61K 31/357; A61K 31/165; A61P 1/00; A61P 1/18

(52) U.S. Cl. 514/452; 514/627

(57) ABSTRACT

The present invention is directed to the treatment of chronic pain that occurs in chronic disease such as chronic pancreatitis by the application, of the drug, resiniferatoxin, to a visceral organ, such as the stomach, which then reduces the pain present in a second visceral organ, such as the pancreas. The reduction of said pain associated with the chronic disease occurs through the drug-induced desensitization of primary pain-sensing neurons that are shared between the two visceral organs. Additionally, application of the drug resiniferatoxin into the first organ, reduces the inflammation that occurs in the second diseased organ. This modulation of shared nerve activity by application of resiniferatoxin, may also be directed to modulate the pain and inflammation in any two visceral organs that share a common spinal or vagal nerve. Finally, application of the drug resiniferatoxin to either the stomach or the jejunum may be used to treat disease and disorders that involve gastroparesis. The resiniferatoxin improves gastric emptying and reduces nausea, thus may be effective therapy for these conditions.

### CROSS-REFERENCE TO RELATED APPLICATIONS

This continuation application claims benefit of priority under 35 U.S.C. §120 of pending international application PCT/US2008/011890, filed Oct. 17, 2008, which claims benefit of priority under 35 U.S.C. §119(e) of provisional application U.S. Ser. No. 60/988,409, filed Oct. 18, 2007, now abandoned, the entirety of both of which are hereby incorporated by reference.

### BACKGROUND OF THE INVENTION

1. Field of the Invention

The present invention relates generally to the fields of therapeutic medicine and pain alleviation. More specifically, the present invention involves the use of a drug in the treatment of chronic pain and inflammation and its use in treating disease or disorders such as chronic pancreatitis and gastroparesis.

2. Description of the Related Art

Excessive alcohol consumption is a common cause of acute and chronic pancreatitis. Further, patients with chronic pancreatitis who continue to drink are most likely to suffer pain exacerbation. The mechanism by which alcohol mediates either inflammation or pain in pancreatitis remains relatively obscure. In this context, most studies have focused on the direct effects of alcohol on pancreatic parenchymal cells or its ability to suppress the immune system with indirect effects on the pancreatic cellular response.

In recent years, it is becoming apparent that visceral nerves, either vagal or spinal in origin, may significantly modulate inflammation in various organs including the pancreas, in a process called “neurogenic inflammation”. However, until now there has been little research on the role that this process plays in mediating the effects of alcohol on the pancreas. Research has examined the effects of acute and chronic pancreatitis on spinal nerves and nociceptive sensitization, particularly focusing on the vanilloid ion channel, transient receptor potential vanilloid type-1 (TRPV1), a key transducer of noxious environmental stimuli. Additionally, local release of the neurotransmitter, substance P, may contribute to a model of acute pancreatitis.

The treatment of pain in chronic pancreatitis remains difficult. Methods such as local application of resiniferatoxin are evolving to treat other forms of chronic pain such as arthritis. The rationale behinds its use in this condition is that resiniferatoxin when instilled locally in the joint, desensitizes pain-carrying nerves (nociceptors) bearing the Transient Receptor Potential Vanilloid Type-1 receptor, for which resiniferatoxin has a high affinity. In theory, this is also an attractive method to treat the pain of pancreatitis, by instillation into the pancreatic duct. While this is possible using endoscopic procedures, such as Endoscopic Retrograde CholangioPancreatography (ERCP), it carries risks of further inflaming the pancreas and requires a high degree of expertise beyond that possessed by the average gastroenterologist. Therefore, there is a need for a safer and simpler method to ablate or desensitize pancreatic nerves.

Thus, the prior art is deficient in direct, safe and simple methods to modulate pain that occurs in an individual diagnosed with a disease or disorder. The present invention fulfills this long-standing need and desire in the art.

### SUMMARY OF THE INVENTION

The present invention is directed to a method to treat pain and inflammation that occurs in an individual due to disease. This method comprises the application of the drug, resiniferatoxin or its derivatives, into an organ not affected by the disease, which reduces pain in a nearby diseased organ. The pain is reduced by desensitization of one or more dichotomous nerve(s) that innervate both the diseased organ and the non-diseased organ.

The present invention is further directed to a method of treating a disease or disorder in an individual, that causes paralysis of the stomach, such as gastroparesis, by the application of the drug, resiniferatoxin or its derivatives, which acts to increase gastric activity and reduce nausea in the individual.

### BRIEF DESCRIPTION OF THE DRAWINGS

FIG. 1 shows a schematic diagram of the proposed role of transient receptor potential vanilloid type-1 (TRPV1) in a model of cerulein-induced pancreatitis. Hyperstimulation of the pancreas with cerulein causes intra-pancreatic activation and release of digestive enzymes. Subsequent tissue acidification (↑H+) or release of an endogenous TRPV1 agonist (↑?) stimulates TRPV1 on primary sensory neurons. TRPV1 activation causes neuronal depolarization and the release of substance P in both the spinal cord and pancreas. Substance P in the pancreas causes tissue edema, neutrophils infiltration and necrosis. Antagonism of TRPV1 with capsazepine does not prevent pancreatic enzyme release yet blocks TRPV1 and limits pancreatic inflammation (Liddle 2007).

FIG. 2 shows the proposed mechanisms of innervation for two visceral organs, the stomach, and the pancreas. On the left, the anatomy of the dorsal root reflex mechanism is shown where a noxious stimulus in the stomach results in an action potential in an afferent nerve fiber that is carried to its central terminal in the dorsal horn. Here, via a spinal interneuron, this potential activates the central terminal of a different afferent nerve fiber coming from the pancreas. This leads to an antidromic current that travels backwards and causes release of neuropeptides locally in the pancreas. On the right is shown the second mechanism where the visceral afferent nerve is dichotomized so that a single dorsal root ganglion neuron provides peripheral branches to both the stomach and pancreas. Irritation of the stomach could then lead to an axonal reflex that results in neurogenic inflammation in the pancreas.

FIG. 3A-3B demonstrates anatomical evidence for a direct neural connection between the stomach and the pancreas. FIG. 3A shows the percentage of pancreatic (dark gray) and stomach (light gray) dorsal root ganglion neurons that were double-labeled with cholera toxin subunit B-594 (dark gray) and cholera toxin subunit B-488 (light gray) on thoracic segments T6-T13 by injection of two distinct retrograde transported neuronal dyes. FIG. 3B is a photomicrograph that demonstrates the presence of gastropancreatic nerves, which are labeled with pancreatic (left photo) and gastric (middle photo) labeling, here shown in an overlay of pancreatic- and gastric-labeled photomicrographs in the right photo.

FIG. 4 shows that gastropancreatic nerves also express the nociceptive receptor, transient receptor potential vanilloid type-1 (TRPV1). The micrograph (1) identifies neurons that express the transient receptor potential vanilloid type-1. The micrograph (2) identifies neurons that are labeled with pancreatic neuronal marker, cholera toxin subunit B-594 (dark gray). The micrograph (3) identifies neurons that are labeled with gastric neuronal label, cholera toxin subunit B-488 (light gray). The micrograph (4) is an overlay of the triple-labeled neurons demonstrating that these neurons are dichotomous and nociceptive in nature.

FIG. 5 shows that activation of gastropancreatic neural reflexes induces pancreatic injury as measured by an increase in pancreatic edema. Capsaicin was infused into the stomach after pyloric ligation and 30 minutes later the pancreas was harvested and water content was measured.

FIG. 6 shows that intragastric alcohol sensitizes pancreatic nerves. A significant increase (P<0.05) in triggered axonal reflexes as measured by multi-unit spike potentials was seen after instillation of alcohol in the stomach and intra-pancreatic capsaicin as compared with the group that received stomach alcohol and intra-gastric saline. Capsaicin dose was 0.05 mg/0.3 ml in 40% ethanol for each group (n=8 rats/group).

FIG. 7 shows alcohol exacerbates pain behavior in a rat model of chronic pancreatitis. Increasing amounts of electrical current were applied to the pancreas through a previously implanted electrode and the total number of nocifensive behaviors was recorded. The alcohol-dosed group showed a significant increase in pain behavior compared to the control (P<0.001 2-way ANOVA, n=9 rats in ethanol group, n=10 rats in saline group).

FIG. 8 shows gastropancreatic nerves can be functionally ablated by application of resiniferatoxin (RTX) into the stomach. Intragastric resiniferatoxin results in near complete attenuation of afferent nerve signaling to colorectal distention at the pressures shown. The top row represents the baseline response. Note the near complete absence of responses 4 hours after intragastric resiniferatoxin.

FIGS. 9A-9B show that local application of resiniferatoxin in either the stomach or in the jejunum accelerates gastric emptying. FIG. 9A shows that instillation of resiniferatoxin into the stomach five weeks prior produces a significant increase in the percentage of gastric emptying after 3 hours compared to the control (P=0.01, n=4 rats). FIG. 9B shows that instillation of resiniferatoxin into the jejunum five weeks prior produces a significant increase in the percentage of gastric emptying after 3 hours compared to the control (P=0.04, n=4).

FIG. 10 shows a model of sensitization of the pancreas to injury by an agent such as cerulein, through a novel gastropancreatic neural reflex mediated by the neuropeptide substance P and its receptor NK1R.

### DETAILED DESCRIPTION OF THE INVENTION

In one embodiment of the present invention, there is provided a method for the treatment of chronic pain and inflammation by the application of resiniferatoxin or its derivatives, into one organ that is not the source of the pain, and which shares one or more pain-sensing nerve(s) with a second organ that is the source of pain, to desensitize the pain-sensing nerves in the second organ.

In another related embodiment of the present invention, there is provided a method of preventing or treating a pathophysiological condition in an individual. Such a method comprises administering the drug resiniferatoxin or its derivatives to one organ that is not involved in the disease or pathophysiological condition, wherein the drug desensitizes the pain response and reduces the inflammation in a second organ that is involved in the disease or pathophysiological condition of said individual, thereby treating the pathophysiological condition in said individual.

In another related embodiment of the present invention, there is provided a method of preventing or treating a pathophysiological condition involving nausea and gastric emptying in an individual, such as occurs in gastroparesis. Such a method comprises applying resiniferatoxin to an organ wherein the drug acts to reduce nausea and aid in gastric emptying, thereby treating the pathophysiological condition in said individual.

As used herein, the term, “a” or “an” may mean one or more. As used herein in the claim(s), when used in conjunction with the word “comprising”, the words “a” or “an” may mean one or more than one. As used herein “another” may mean at least a second or more.

As used herein, the term, “organ” comprises visceral organs located in the thoracic or abdominal cavity, which comprises the pancreas, bladder, stomach, spleen, kidney, urinary tract, gallbladder, heart, liver, lung, esophagus, diaphragm, small intestine, large intestine, colon, bile duct, pancreatic duct, prostate, uterus, thyroid gland, appendix, and male and female sexual organs.

As used herein, the term, “dichotomous nerve” is a nerve that innervates two separate organs by means of branching at its terminal end into two nerves. This branching nerve can sense and send nerve signals between the two organs.

As used herein, the term, “gastropancreatic nerve” is a nerve that forms a branch at its terminal ends such that one branch innervates a visceral organ, the stomach, and the second branch innervates a second visceral organ, the pancreas. As used herein, this gastropancreatic nerve receives input and can send signals between the stomach and pancreas.

As used herein, the term “neurogenic inflammation” is a result of nerve activation that produces arteriolar vasodilatation, increased vascular permeability, edema, and neutrophil infiltration.

As is well known in the art, a specific dose level of such an drug for any particular patient depends upon a variety of factors including the activity of the specific drug employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety, and purity standards as required by Food and Drug Administration Office of Biologics standards.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Alcohol and Pancreatitis

Although alcohol is the most common cause of pancreatitis, much remains unknown about the underlying mechanisms. By itself, chronic alcohol administration to animals results in relatively subtle effects on the pancreas. This has led to the hypothesis that alcohol acts as a permissive factor for acute pancreatitis by “sensitizing” inflammatory and other biological pathways that are invoked by classical trigger factors.

In vitro studies on isolated pancreatic acini strongly suggest that a critical and early step in the pathogenesis of acute pancreatitis is the premature activation of proteolytic zymogens within pancreatic acinar cells (Saluja et al, 2007). Much remains to be known about the precise sequence of events leading to this; however, it is clear that it requires an increase in cytosolic calcium (Gorelick 2003). In addition to a direct effect on zymogen activation, elevated calcium has also been shown to trigger NFkB and other “pro-inflammatory” signaling pathways within the acini (Pandol et al, 2003). In vivo, the story appears to be more complicated. Cerulein-induced pancreatitis has been shown to be associated with a biphasic pattern of trypsinogen activation in the pancreas with the early peak after one hour and the second peak several hours later (Luthen et al, 1995). The cause of these two peaks may differ, and it has been suggested that the latter is dependent on inflammation (Lerch et al, 2003). Inflammation has been shown to be necessary to invoke zymogen activation and pancreatic cell death in response to high-doses of cerulein, an effect that requires activated neutrophils and their ability to generate reactive oxygen species (ROS) via the enzyme nicotinamide adenine dinucleotide phosphate- (NADPH) oxidase (Gukovskaya et al, 2002).

The role of alcohol in acute pancreatic injury may involve several mechanisms. In the presence of alcohol, low-dose cholecystokinin activates zymogens in pancreatic lobules much more efficiently (by a six-fold factor), reaching levels similar to that achieved otherwise only with high doses (Katz et al, 1996). There are several ways by which alcohol has been proposed to induce such sensitization, many of which converge on changes in the level of intracytosolic calcium or protein kinase C activation. In vitro, alcohol augments cholecystokinin-induced rises in intracytosolic calcium as well making the zymogen granules more responsive to the effects of sustained elevations of calcium (Ding et al, 2006). Further, alcohol along with submaximal cholecystokinin can mimic the deleterious effects of supramaximal cholecystokinin on intracellular trafficking of enzymes by diverting exocytosis from apical to basolateral plasma membrane sites, in a manner that is protein kinase C dependent and requires the participation of Munc-18c and various specific components of the intra-acinar SNARE complex (Cosen-Binker et al, JBC, 2007; Cosen-Binker et al, Gastroenterol, 2007; Cosen-Binker et al, Can J Gastroenterol, 2007). Alcohol also sensitizes the pancreas to injurious stimuli in vivo. A significant inflammatory response in the pancreas is seen in animals chronically fed alcohol and given an otherwise innocuous low-dose cholecystokinin infusion (Pandol, et al 1999). In this context, ethanol treatment enhances Protein kinase C activation of NFkB (Gukovskaya et al, 2004).

### TRPV1 and Pancreatitis

Primary nociceptive sensory neurons consist predominantly of slow unmyelinated C-fibers and some fast myelinated Ad fibers; these are polymodal in that they respond to both mechanical and thermal stimuli, with C-fibers also responding to noxious chemical stimuli such as acid and capsaicin. Most visceral C-type nociceptors contain neuropeptides such as substance P (SP) and calcitonin gene-related peptide (CGRP) and are dependent on trophic support by nerve growth factor (NGF) (Millan, 1999). Nociceptors convert noxious stimuli to an electrical response via specialized receptors of which the most important is transient receptor potential vanilloid type-1 (TRPV1) (Pingle et al, 2007; Immke et al, 2006; Szallasi et al, 2006; Suh and Oh, 2005; Tominaga and Tominaga, 2005; Jia et al, 2005; Planells-Cases et al, 2005; Holzer et al, 2004; Nagy et al, 2005). In recent years, it has become apparent that the communication between primary nociceptor neurons and the pancreas is actually bidirectional, with the nerves both responding to and modulating changes in their environment. Thus activation of the nerve endings of these neurons results in arteriolar vasodilatation, increased vascular permeability, edema, and neutrophil infiltration, collectively termed “neurogenic inflammation” (Planells-Cases et al, 2005; Levine et al, 2006; Zegarska et al, 2006; Schaible et al, 2005; Garle and Fry, 2003; Richardson and Vasko, 2002). This process has been shown to be important in the pathogenesis of acute pancreatitis as well (Liddle, 2007; Vera-Portocarrero and Westlund, 2005; Liddle and Nathan, 2004; Hegde and Bhatia, 2005). Thus functional denervation of the pancreas by neonatal capsaicin treatment, celiac ganglionectomy or resiniferatoxin (a ultrapotent capsaicin analog that produces desensitization of nociceptors) results in significant attenuation of acute experimental pancreatitis (Nathan et al, 2002; Noble et al, 2006).

### The Capsaicin Receptor, Transient Receptor Potential Vanilloid Type-1 (TRPV1)

As referenced supra, nociceptors convert noxious stimuli to an electrical response via specialized receptors such as the vanilloid receptor (TRPV1 or VR1). This receptor is expressed by nociceptive primary afferents, along with neuropeptides such as substance P and calcitonin gene-related peptide in primary nociceptors. TRPV1 responds to and appears to integrate several noxious stimuli produced during tissue injury, including heat, local tissue acidosis, and several pro-algesic metabolites. Activation of the receptor results in a cationic, calcium-preferring current, which leads to depolarization of the membrane. Acid and heat are both thought to function as endogenous ligands of this receptor and recent evidence also points to a potential role for other biologically-active compounds such as anandamide and related lipid metabolites.

TRPV1 activation/gating has several potential consequences. The depolarizing current can be propagated orthodromically to the spinal terminals of the nociceptor, where it is relayed further up the pain-sensing pathway. On the other hand, antidromic propagation to collateral nerve fibers results in the local release of neuropeptides, including calcitonin gene-related peptide (CGRP), the tachykinins, substance P (SP) and neurokinin A (NKA). As has been shown in other organs, this sequence of events can also induce neurogenic inflammation in the pancreas. Thus capsazepine, a TRPV1 antagonist can attenuate cerulein induced pancreatitis in mice via NK1R activation (Huller et al, 2005). Furthermore, capsazepine, a selective TRPV1 antagonist, can reduce cerulein-induced pancreatitis in mice, along with reduction of NK1R endocytosis in acinar cells, a surrogate marker for SP release (Nathan et al, 2001).

### Substance P

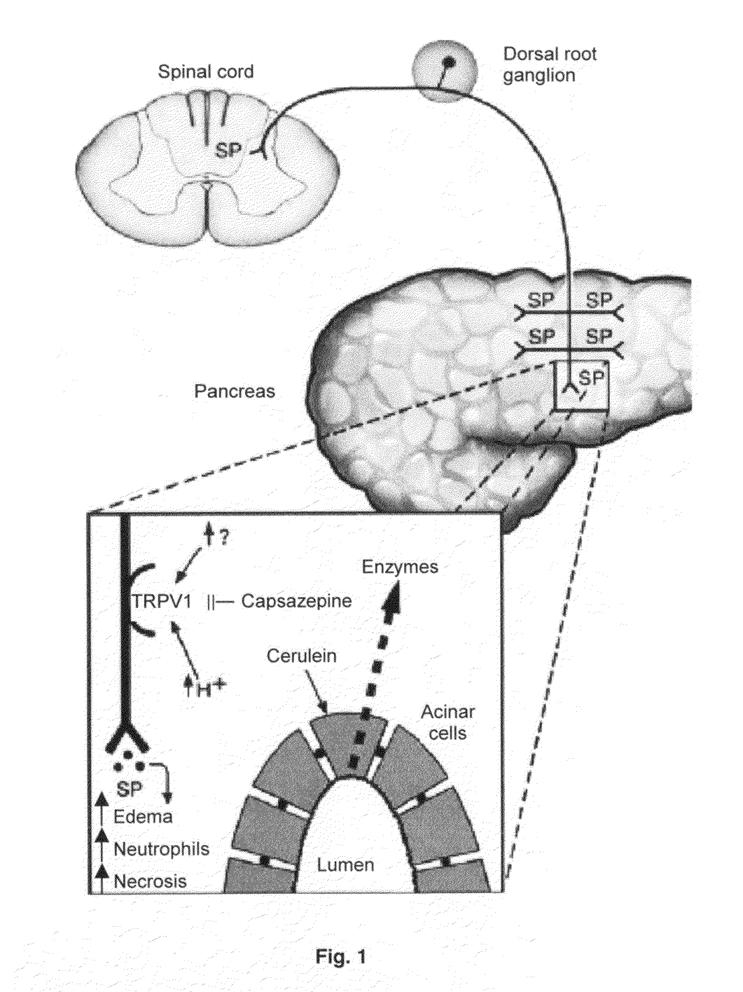

Substance P (SP), a product of the larger tachykinin family, is a product of the tachykinin 1 gene (Tac 1, preprotachykinin/PPT-A) and has diverse biological effects mediated by one of three neurokinin receptors, NK1-3. Of these the neurokinin-1 receptor (NK-1R), a G-protein coupled receptor, is particularly important in tissue inflammation. According to the neurogenic paradigm of inflammation, SP release from nerves leads to the activation of NK1R in a variety of cells including immune cells, endothelial cells and parenchymal cells. Pancreatic levels of SP are elevated in cerulein-induced inflammation in mice, along with upregulation of NK-1 receptors on the acinar cells in the pancreas (Bhatia et al, 1998). In the pancreas, this leads to changes in vascular permeability, edema, neutrophil adhesion migration and accumulation in the pancreas (Hutter et al, 2005; Ito et al, 2007). Neurokinin signaling can also stimulate enzyme secretion (Sjodin et al, 1994; Sjodin et al, 1990; Sjodin et al 1991), as well as chemokine synthesis by pancreatic cells (Ramnath and Bhatia, 2006; Sun and Bhatia, 2007). Genetic disruption of either preprotachykinin or NK-1R expression protects against both pancreatic and associated lung injury (Bhatia et al 1998; Bhatia et al, 2003), and is an important determinant of lethality in experimental pancreatitis (Maa et al 2000). Conversely, pancreatitis-associated injury is worse in mice with deletion of neutral endopeptidase, the enzyme responsible for degrading extracellular SP (Day et al, 2005). Thus a model emerges in which TRPV1 activation on peripheral nerves results in release of substance P, activation of NK1R and inflammation (shown in FIG. 1).

### Alcohol and Pain in Chronic Pancreatitis

Pain is the cardinal feature of chronic pancreatitis from almost any cause and occurs in 75-90% of patients with alcoholic chronic pancreatitis (DiMagno, 1999; Ammann, 1997). Although much needs to be learned about the mechanisms of pain in these patients, it is clear that ongoing alcohol intake is a major factor affecting the frequency and severity of pain. Pain relief is more likely to occur in patients who have stopped drinking. A study showed that complete relief of pain occurred in nearly 60% of abstinent patients as compared with only 33.3% of those who continued to drink (Gastard et al, 1973); this is similar to a study that showed ongoing alcohol consumption was significantly higher in the pain group (70.4% versus 35% of the no pain group; p<0.002) (de las Heras et al, 1995). The influence of alcohol on pain may be independent of its effect on progression of pathological changes, which can occur even despite abstinence (Ammann et al 1984; Sarels, 1985).

### TRPV1 and Pain in Chronic Pancreatis

The pathogenesis of pain in chronic pancreatitis remains to be fully understood. Recent attention has been focused on the effects of chronic pancreatitis on pancreatic nerves and associated changes in the expression of key molecular mediators of signaling (Bornman et al, 2003; Di Sebastiano et al, 2003; Friess et al, 2002; Zhu et al, 2001; Shrikhande et al, 2001; Berberat et al, 2000, Di Sebastiano et al, Gut, 2000; Di Sebastiano et al, Ann Rai Chir, 2000; Kleeff et al, 2000; Friess et al, 1999). A rat model of chronic pancreatitis was developed to study pain by using intraductal trinitrobenzene sulfonic acid (TNBS) and shown to display construct, face and predictive validity (Winston et al, 2005). This model has been successfully used to demonstrate that chronic pancreatitis is associated with upregulation of both SP and CGRP in spinal afferents in a pancreas-specific distribution and suppression of Kv currents in pancreas-specific nociceptors (Xu et al, 2006). Chronic pancreatitis causes a four-fold increase in capsaicin-induced current density (P<0.02), along with an increase in proportion of pancreas-specific dorsal root ganglion neurons that responded to capsaicin (52.9% in controls versus 79.0% in CP; P<0.05) (Xu et al, 2007). Chronic pancreatitis was also associated with a significant increase in TRPV1 expression both at the mRNA and protein levels in pancreas-specific sensory neurons. Systemic administration of the TRPV1 antagonist, SB-366791, markedly reduced the pancreatic pain behavioral response. These results suggest that TRPV1 upregulation and sensitization is a specific molecular mechanism contributing to the hyperalgesia in chronic pancreatitis.

### Alcohol and TRPV1

There is evidence to implicate a role for both alcohol and TRPV1 in the pathogenesis of pancreatic inflammation and pain. What is not known is if these two factors interact. In vitro, ethanol can directly stimulate the TRPV1 receptor as measured by a capsazepine-sensitive calcium response in trigeminal and dorsal root ganglia neurons; more importantly perhaps, ethanol can also sensitize dorsal root ganglia neurons to the effects of capsaicin (refer to FIG. 1) (Trevisani et al, 2002). Finally, alcohol can also induce the release of substance P from various tissues in a capsazepine-sensitive manner, again suggesting the involvement of TRPV1. The stimulation of TRPV1 by alcohol can explain the typical burning sensation experienced when ingesting a high concentration of alcohol or when alcohol is applied to the skin.

A role for alcohol in neurogenic inflammation has been suggested recently in an experimental model of reactive airway disease. Alcohol caused contractions of ex-vivo preparations of guinea pig bronchi in a manner that was sensitive to both TRPV1 and tachykinin receptor blockade. In vivo, alcohol by either the intravenous (221 mg/kg) or the intragastric (790 mg/kg) route of administration caused broncho-constriction and increased plasma extravasation in the guinea pig airways in a capsazepine-sensitive manner (Trevisani et al, 2004).

### Example 2

Information Sharing by Means of Shared Innervation Between Two Visceral Organs

Although the pancreas is not directly exposed to orally ingested alcohol, there are several ways in which alcohol can access TRPV1 in peripheral nerves and initiate neurogenic inflammatory and nociceptive signaling in this organ. The most obvious is via circulating alcohol absorbed from the gastrointestinal tract. However, it is not clear whether circulating levels after modest drinking can reach and sustain those required. An alternative and novel pathway may involve neural reflexes originating in neighboring organs such as the stomach or duodenum, which do get exposed to high alcohol concentrations after drinking. This is an example of viscero-visceral convergence.

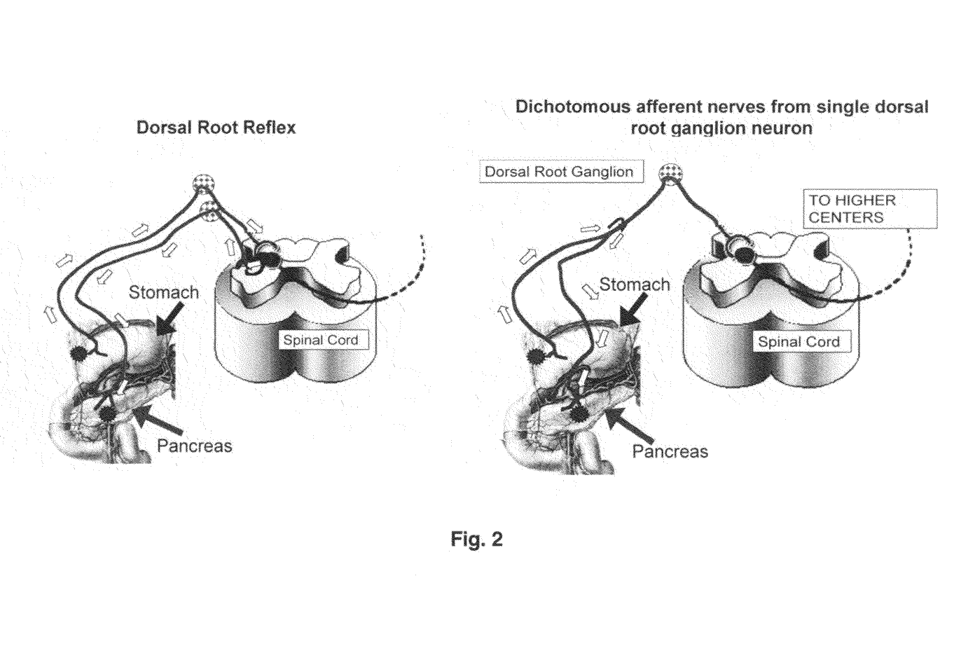

Convergence of information from two or more visceral organs can take place at several levels in the peripheral central nervous system including the spinal cord and higher centers. This is important in coordinating physiological events such as urination, defecation, and coitus, which should normally inhibit each other. This process also may play a role in inflammation and pain and provides a mechanism by which disease in one visceral organ induces pathology in another (Pezzone et al, 2005). Thus, acute cystitis in an otherwise health bladder can be observed in response to colitis, prostatitis, or endometriosis in rats (VVinnard et al, 2006). Chronic colonic inflammation produces neurogenic inflammation in the bladder accompanied by disturbed passage of urine (micturition), recruitment, and activation of bladder mast cells, and up regulation of neurotrophic growth factor (NGF) and stem cell factor (SCF) in the bladder (Liang et al, 2007; Ustinova et al, 2007; Ustinova et al, 2006). Further, acute colitis can lead to up regulation of sodium currents in primary sensory neurons emanating from the bladder (Malykhina et al, 2004). The increased vascular permeability in the bladder in response to inflammation in the colon or uterine horn can be reduced by hypogastric nerve ablation implying the existence of hardwired neural pathways (Winnard et al, 2006). What is not clear is the nature of this hardwiring. Several mechanisms are possible, of which only two are shown in FIG. 2.

The first mechanism is the dorsal root reflex (shown on the left in FIG. 2) in which a noxious stimulus in the stomach results in an action potential in an afferent fiber that is carried to its central terminal in the dorsal horn. Here, via a spinal interneuron, it activates the central terminal of a different afferent fiber coming from the pancreas. This leads to an antidromic current that travels backwards and causes release of neuropeptides locally in the pancreas. The second mechanism is dichotomizing visceral afferents, where a single dorsal root ganglion neuron provides peripheral branches to both stomach and pancreas (shown on the right in FIG. 2). Irritation of the stomach could then lead to an axonal reflex that results in neurogenic inflammation in the pancreas. There is considerable evidence for such pathways to exist in other organs, in addition to the stomach and pancreas. Using both anatomical and physiological techniques, axon dichotomy has been found to be a property of dorsal root ganglion neurons in many species, ranging from 0.5 to 15% of all afferents (McNeill and Burden, 1986). This has been particularly of interest in the pelvic and lower visceral organs (Keast and De Groat, 1992). Twenty-one percent of mice and up to 17% of rat urinary bladder and colon afferents are thought to be dichotomized based on dual labeling studies (Christianson et al, 2007). Definitive proof of this phenomenon has come from single unit recordings of visceral afferents, which show responses to stimulation of both the lower urinary tract and the colon (Bahns et al, 1987).

### Example 3

### Anatomical Evidence Demonstrates a Direct Neural Connection Exists Between the Stomach and Pancreas, Wherein a Subset of Afferent Neurons Innervates Both the Stomach and Pancreas Via Dicohotomous Peripheral Branches

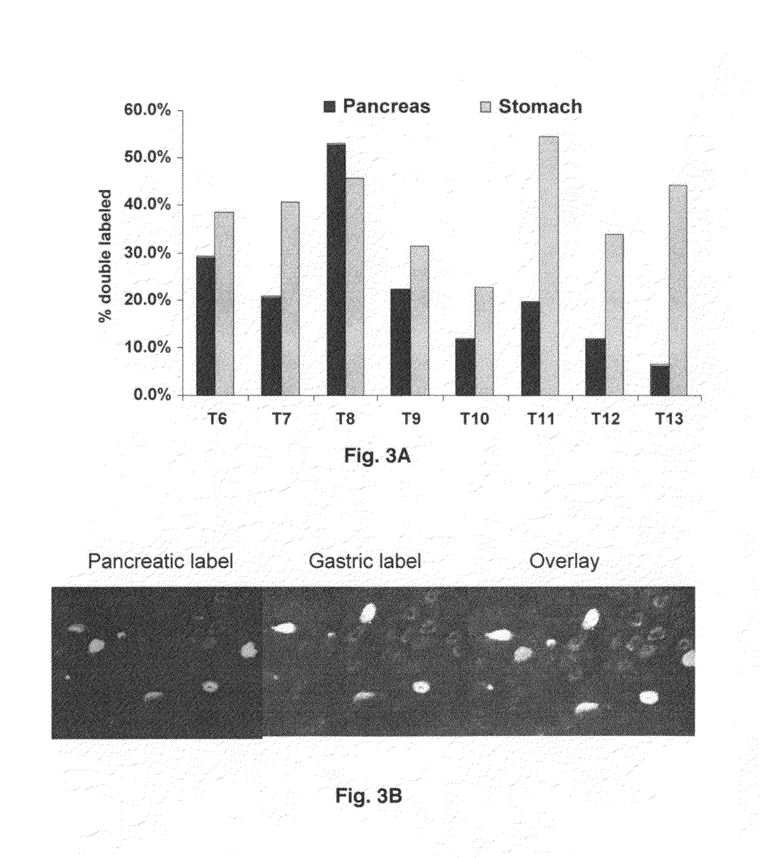

Based on the hypothesis that irritation of the stomach could affect the pancreas, the possibility of neural convergence in these two neighboring organs was first examined, specifically focusing on dichotomizing branches of afferent neurons. This was examined by injection of two distinct retrograde transported neuronal dyes into the pancreas, cholera toxin subunit B-594 (red-label) (Invitrogen; 24 ml in 12 sites, 2 ml site at 2 mg/ml) and in the stomach, cholera toxin subunit B-488 (green), at the same concentration using the same injection strategy by injection into the muscle wall and under the serosa of rats. Five days later, the animals were euthanized and the dorsal root ganglions from thoracic segments T6-T13 were harvested and sectioned. The percentage of single-labeled and double-labeled neurons was counted and shown in FIG. 3A. The number of double-labeled cells is expressed as a percentage of the total number of gastric- and pancreatic-labeled cells. Out of 2,333 dorsal root ganglion neurons examined, 1,779 originated from the stomach, and 898 neurons originated from the pancreas, and 344 originate from both organs. Overall, 19% of all pancreatic cells also express gastric-labeling while 38% all gastric cells also expressed pancreatic labeling. Additionally, at T8 more than half of all pancreatic segments are also innervated from the stomach. Similarly, in T10 more than 50% of all gastric neurons also innervate the pancreas. Thus, the term “gastropancreatic nerve” is used to identify nerves that receive input from both the stomach and pancreas. A representative photomicrograph of double labeled gastropancreatic nerves is shown in FIG. 3B.

### Example 4

### Most Gastropancreatic Nerves are Nociceptive in Nature as Revealed by Expression of Characteristic Markers

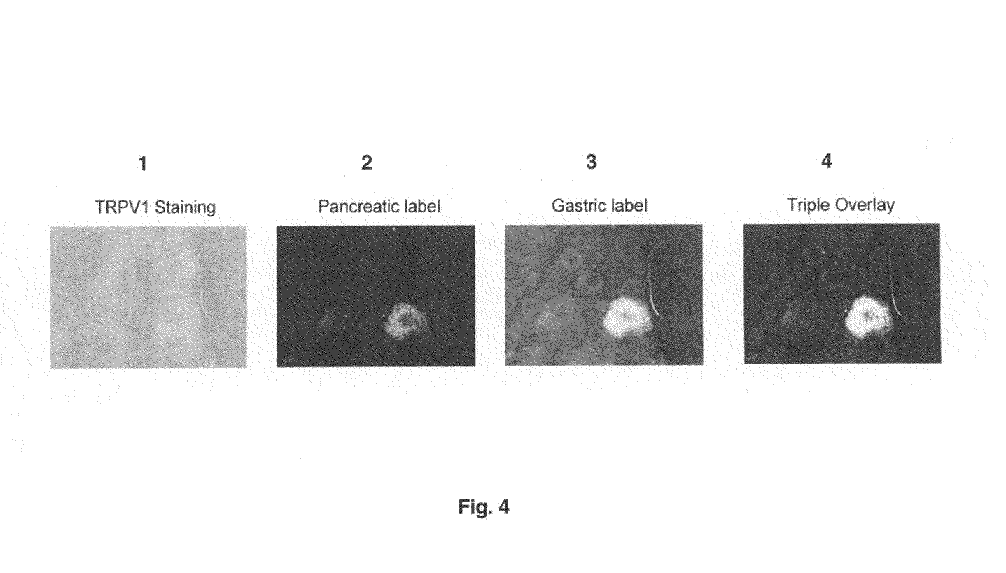

As shown in Table 1, most gastropancreatic nerves expressed markers for nociceptive neurons equally or more frequently than single-labeled nerves. Nociceptor neurons are considered either peptidergic or in origin. Calcitonin gene-related peptide (CGRP) is a commonly used marker for the peptidergic-type neuron, and IB4 for the non-peptidergic-type neuron. There is, however, considerable overlap as shown in Table 1. Both transient receptor potential vanilloid type-1 (TRPV1) and P2X3 (a purinergic G-protein coupled receptor) are highly characteristic of nociceptors and are very important molecules in transducing tissue injury into electric signals. An example of transient receptor potential vanilloid type-1 expression in a gastropancreatic neuron is shown in FIG. 4.

TABLE 1
Most gastropancreatic nerves are nociceptive in nature.

| | Pancreas | Stomach | Both |
|---|---|---|---|
| CGRP | 68% | 79% | 85% |
| IB4 | 59% | 50% | 67% |
| P2X3 | 42% | 56% | 64% |
| TRPV1 | 73% | 78% | 71% |

### Example 5

### Activation of Gastropancreatic Neural Reflexes Induce Pancreatic Injury

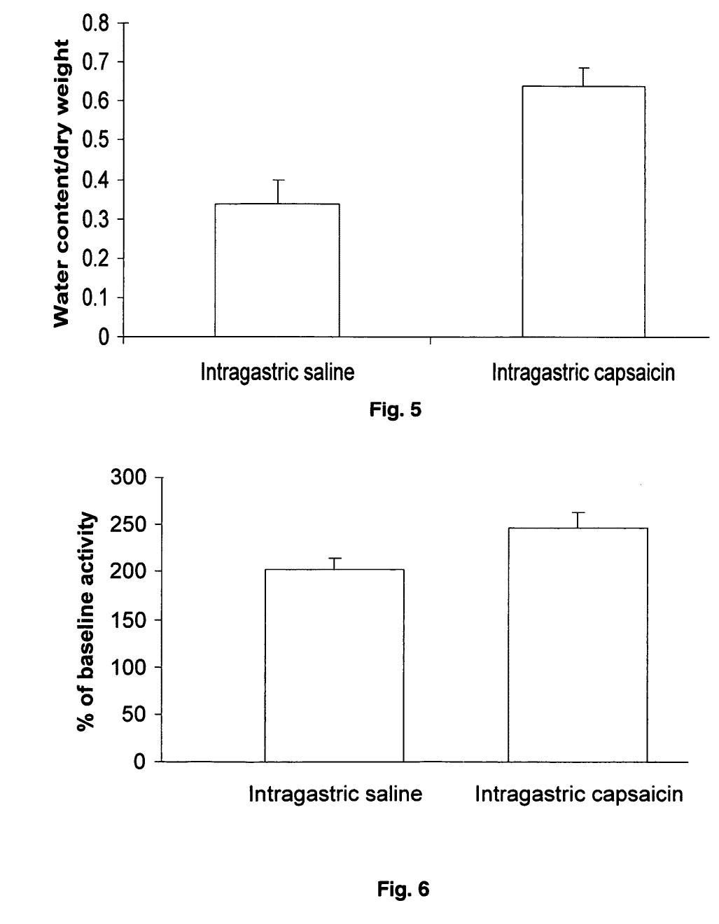

Next, the functional correlate of this anatomical convergence was determined. It was hypothesized that intragastric capsaicin will activate transient receptor potential vanilloid type-1 on gastric branches of dichotomous gastropancreatic nerves and via an axonal reflex result in edema of the pancreas. After pyloric ligation, intragastric infusion of 1.2 ml of 1.6 μM capsaicin was followed 30 minutes later by harvesting of the pancreas and measurement of water content. The results show that intragastric capsaicin causes pancreatic edema (FIG. 5).

### Example 6

### Intragastric Alcohol Sensitizes Gastropancreatic Nerves

Transient receptor potential vanilloid type-1 activation on the gastric side of gastropancreatic nerves can trigger axonal reflexes. Based on the known interaction between alcohol and transient receptor potential vanilloid type-1, it is possible that intragastric ethanol might also stimulate these nerves. Hence, ethanol was instilled in the stomach via an orogastric lavage, after pyloric ligation to limit systemic absorption from the intestine (only 10% of alcohol is absorbed from the stomach) (Levitt et al, 1997). At the same time, there was an intra-pancreatic canula in place and recording electrodes positioned around nerve fibers in the greater splanchnic nerve that were responsive to mechanical stimulation of the pancreas. Five minutes after intragastric ethanol, capsaicin was infused into the pancreatic duct. Ethanol by itself did not increase gastropancreatic nerve activity, however a significant increase in multi-unit spike potentials was seen after intra-pancreatic capsaicin as compared with the group that received intragastric saline (FIG. 6), proving that intragastric alcohol sensitizes pancreatic afferent neurons to chemical stimulation.

These findings strongly support the hypothesis that intragastric ethanol activates transient receptor potential vanilloid type-1 on gastropancreatic nerves and sets up axonal reflexes, that in turn affect pancreatic branches, lowering their threshold for stimulation. Of note, efferent function in nociceptor nerves can occur at thresholds of excitation that are below those required to evoke classic action potentials and othrodromic signaling (Szolcsanyi, 2004). This may account for why intragastric ethanol causes no change in the baseline rate of firings.

### Example 7

### Alcohol Exacerbates Pain Behavior in a Model of Chronic Pancreatitis

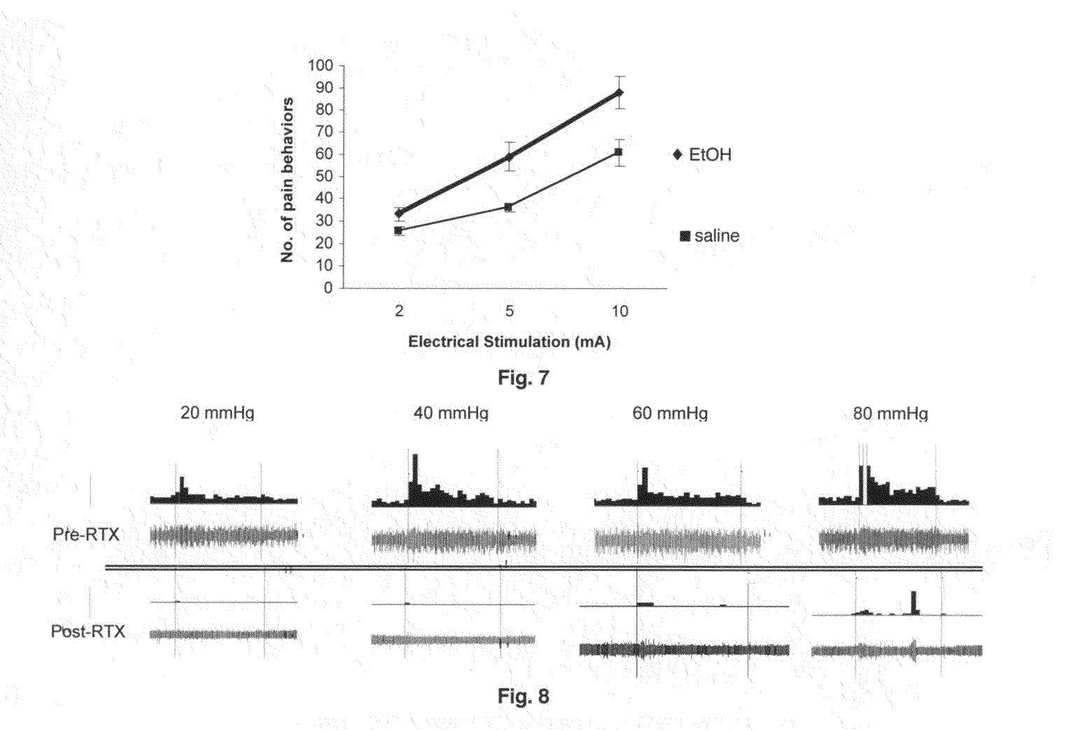

Adult male rats (˜400 gms) 3-4 weeks post trinitrobenzene sulfonic acid (TNBS) treatment were administered with 20% ethanol (1.6 gm/kg body weight) in normal saline (1.3 ml) 3 times a day at hourly intervals for 3 consecutive days. Control TNBS rats received an equal volume of normal saline. The next day the rats were tested for their behavioral response for pain by administering successive application of electrical current at 2, 5 and 10 mA for 5 minutes through a pair of electrodes implanted in the pancreas at the time of TNBS administration. The total number of nocifensive behaviors during this stimulation period was recorded and analyzed (FIG. 7). The results demonstrate that alcohol exposure increased the number of nocifensive behaviors significantly (P<0.001 using 2-way ANOVA).

### Example 8

### Gastropancreatic Nerves Functionally Ablated by Intragastric Resiniferatoxin

Resiniferatoxin is the principal active ingredient in the classic drug Euphorbium, the air-dried latex (resin) of the cactus-like plant Euphorbia resinifera. It is an ultra-potent analog of capsaicin and local application causes desensitization without prior excitation of afferent nerves that lasts for several weeks (Neubert et al, 2003; Kissin et al, 2002; Xu et al, 1997; Craft et al, 1995; Avelino et al, 1999; Acs et al, 1997; Abdel-Salam et al, 1994). In this regard, resiniferatoxin is 200 times more potent than capsaicin, and is an effective tool for causing selective site-specific desensitization to ablate both pain responses and neurogenic inflammation. As a proof of principle, this ability was examined in the rat stomach and intragastric resiniferatoxin results in nearly complete attenuation of afferent nerve signaling to colorectal distention (FIG. 8).

### Example 9

### Resiniferatoxin Accelerates Gastric Emptying

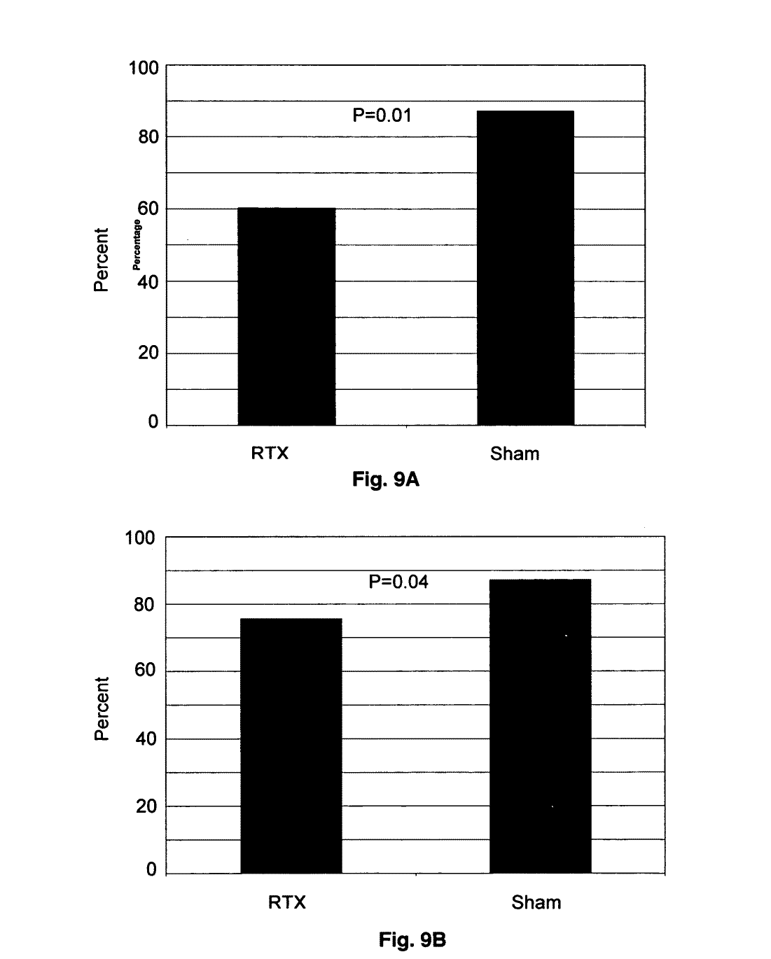

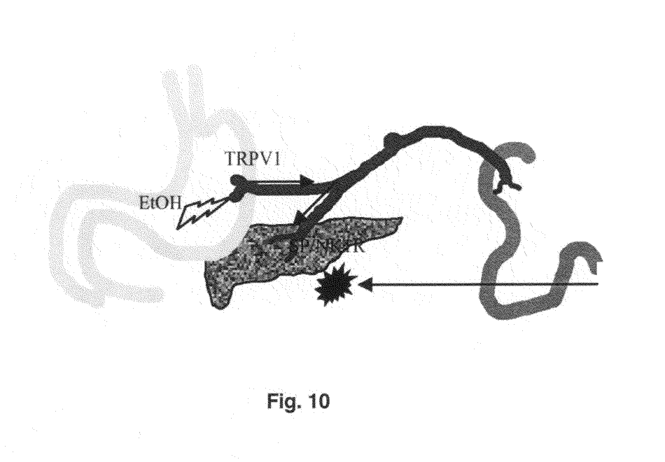

In addition to treating nausea with local instillation of resiniferatoxin it was found that local application of resiniferatoxin into either the stomach or in the jejunum can accelerate gastric emptying. These experiments were done in male rats. Resiniferatoxin was instilled at 100 nM/kg body weight, 0.6 microgm/ml, into either the stomach or jejunum. Controls received saline in the stomach. Five weeks later gastric emptying of solid foods was measured. The results, expressed as the percentage of emptying at 3 hours, are shown in FIG. 9A-9B. The data indicates that local application of resiniferatoxin into either the stomach or into the jejunum accelerates gastric emptying. The combination of faster emptying and anti-nauseant effect makes it a very appropriate treatment for gastroparesis.

The following references were cited herein:
Abdel-Salam O M et al., (1994) J Physiol Paris; 88:353-8.
Acs G, et al., (1997) J Neurosci; 17:5622-8.
Ammann R W, et al., (1984) Gastroenterology, 86:820-8.
Ammann R W. (1997) Pancreas; 14:215-21.
Avelino A, et al., (1999) Eur J Pharmacol; 378:17-22.
Bahns E, et al., (1987) Pflugers Arch; 410:296-303.
Berberat P O, et al., (2000) Swiss Surg; 6:227-30.
Bhatia M, et al., (1998) Proc Natl Acad Sci USA; 95:4760-5.
Bhatia M, et al., (2003) Am J Physiol Gastrointest Liver Physiol; 284:G830-6.
Bornman P C, et al., (2003) World J Surg; 27:1175-82.
Christianson J A, et al., (2007) Pain; 128:235-43.
Cioffi D L, (2007) Circ Res; 100:934-936.
Cosen-Binker L I, et al., (2007) Gastroenterology, 132:1527-45.
Cosen-Binker L I, et al., (2007) J Biol Chem; 282:13047-58.
Cosen-Binker L I, Gaisano H Y, (2007) Can J Gastroenterol; 21:19-24.
Craft R M, et al.; (1995) Pain; 61:317-23.
Day A L, et al., (2005) J Surg Res; 128:21-7.
de las Heras G, et al., (1995) J Clin Gastroenterol; 20:33-6.
Di Sebastiano P, et al., (2000) Ann Mal Chir, 71:11-6.
Di Sebastiano P, et al., (2000) Gut, 47:423-8.
Di Sebastiano P, et al., (2003) Gut 52:907-11.
DiMagno EP. (1999) Gastroenterology, 116:1252-7.
Ding Y X, et al., (2006) Biochem Biophys Res Commun; 350:593-7.
Friess H, et al., (1999) Ann Surg; 230:615-24.
Friess H, et al., (2002) Gut; 50:682-6.
Garle M J, Fry J R. (2003) Altern Lab Anim; 31:295-316.
Gastard J, et al., (1973) Digestion; 9:416-28.
Gorelick F S, (2003) Pancreas; 27:305-10.
Gukovskaya A S, et al., (2002) Gastroenterology, 122:974-84.
Gukovskaya, et al, (2004) Am J Physiol Gastrointest Liver Physiol; 286:G204-13.
Gullo L, et al., (1988) Gastroenterology; 95:1063-8.
He Z J, et al., (2003) Pancreas; 27:e13-7.
Hegde A, Bhatia M. (2005) Jop; 6:417-21.
Holzer P, (2004) Neurogastroenterol Motil; 16:697-9.
Hoogerwerf W A, et al., (2001) J Neurosci; 21:9036-42.
Hoogerwerf W A, et al., (2004) Gastroenterology; 127:883-91.
Hoogerwerf W A, et al., (2005) BMC Gastroenterol; 5:8.
Flutter M M, et al., (2005) Pancreas; 30:260-5.
Immke D C, Gavva N R. (2006) Semin Cell Dev Biol; 17:582-91.
Ito Y, et al., (2007) Pancreas; 34:138-43.
Jia Y, (2005) Drug News Perspect, 18:165-71.
Katz M, et al., (1996) Am J Physiol; 270:G171-5.
Keast J R, De Groat W C, (1992) J Comp Neurol; 319:615-23.
Kissin I, et al., (2002) Anesth Analg; 94:1253-8.
Kleeff J, et al., (2000) Ann Hal Chir; 71:3-10.
Lerch M M, et al., (2003) Pancreas; 27:291-6.
Levine J D, et al., (2006) Ann N Y Acad Sci; 1069:155-67.
Levitt M D, et al., (1997) Alcohol Clin Exp Res; 21:293-7.
Liang R, et al., (2007) Neurourol Urodyn.
Liddle R A, Nathan J D. (2004) Pancreatology. 4:551-9; discussion 559-60.
Liddle R A. (2007) Biochim Biophys Acta: 1772(8): 869-78.
Luthen R, et al., (1995) Am J Physiol; 268:G592-604.
Maa J, et al., (2000) Surgery; 128:232-9.
Malykhina A P, et al., (2004) Neuroreport; 15:2601-5.
McNeill D L, Burden H W, (1986) Anat Rec; 214:441-4.
Millan M J. Progress in Neurobiology 1999; 57:1-164.
Nagy I, et al., (2004) Eur J Pharmacol; 500:351-69.
Nathan J D, et al., (2001) Am J Physiol Gastrointest Liver Physiol; 281:G1322-8.
Nathan J D, et al., (2002) Am J Physiol Gastrointest Liver Physiol; 283:G938-46.
Neubert J K, et al., (2003) Pain; 104:219-28.
Noble M D, et al., (2006) Am J Physiol Gastrointest Liver Physiol; 291:G128-34.
Pandol S J, et al., (1999) Gastroenterology, 117:706-16.
Pandol S J, et al., (2003) J Gastroenterol; 38:623-8.
Pezzone M A, et al., (2005) Gastroenterology 128:1953-64.
Pingle S C, et al., (2007) Handb Exp Pharmacol: 155-71.
Planells-Cases R, et al., (2005) Pflugers Arch; 451:151-9.
Ramnath et al. (2006) Am J Physiol Gastrointest Liver Physiol; 291:G1113-9.
Richardson J D, Vasko M R. (2002) Pharmacol Exp Ther, 302:839-45.
Saluja A K, et al., (2007) Annu Rev Physiol; 69:249-69.
Sarles H. (1985) Scand J Gastroenterol; 20:651-9.
Schaible H G, et al., (2005) Rheum Dis Clin North Am; 31:77-101, ix.
Shrikhande S V, et al., (2001) Pain; 91:209-17.
Sjodin L, et al., (1990) Regul Pept, 30:239-53.
Sjodin L, et al., (1991) P. J Physiol; 444:763-76.
Sjodin L, et al., (1994) J Physiol; 476:69-77.
Suh Y G, Oh U. (2005) Curr Pharm Des; 11:2687-98.
Sun J, Bhatia M. (2007) Am J Physiol Gastrointest Liver Physiol; 292:G143-53.
Szallasi A, Blumberg P M, (1999) Pharmacol Rev: 51:159-212.
Szallasi A, et al., (2006) Trends Mol Med; 12:545-54.
Szolcsanyi J., (2004) Neuropeptides 38:377-384.
Toma H, et al., (2000) Gastroenterology; 119:1373-81.
Toma H, et al., (2002) Pancreas; 25:31-8.
Tominaga M, Tominaga T, (2005) Pflugers Arch; 451:143-50.
Trevisani M, et al., (2002) Nat Neurosci; 5:546-51.
Trevisani M, et al., (2004) J Pharmacol Exp Ther 309:1167-73.
Ustinova E E, (2006) Am J Physiol Renal Physiol; 290:F1478-87.
Ustinova E E, et al., (2007) Am J Physiol Renal Physiol; 292:F123-30.
Vera-Portocarrero L, Westlund K N. (2005) Neurosignals; 14:158-65.
Winnard et al., (2006) Am J. Phys. Regul Integr Comp Physiol; 291:R1592-601.
Winston J H, et al., (2003) J Pain; 4:329-37.
Winston J H, et al., (2005) Pain; 117:214-22.
Xu G Y, et al., (2006) Am J Physiol Gastrointest Liver Physiol; 291:G424-31.
Xu G, et al., (2007) Gastroenterology: (In press).
Xu X J, et al., (1997) Brain Res; 752:52-60.
Zegarska B, et al., (2006) Pharmacol Rep; 58:13-21.
Zhang L L, et al., (2007) Circ Res; 100:1063-1070.
Zhu Z W, et al., (2001) Dig Dis Sci; 46:1633-9.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A method of treating pain and inflammation in an organ of an individual comprising:
applying a drug to one organ not causing said pain, and
desensitizing the shared pain-sensing nerves present between the one organ not causing said pain and a second organ, that is the source of said pain and inflammation, through said application of said drug, wherein said desensitizing reduces the pain and inflammation present in the second organ through said application of said drug.

2. The method of claim 1, wherein the sharing of pain-sensing nerve(s) present between the two organs is through one or more dichotomous nerve(s) that is branched into two ends which terminate in two separate organs.

3. The method of claim 2, wherein said dichotomous nerve(s) is a pain-sensing nerve, composed of slow and fast nerve fibers, that is responsive to mechanical, thermal and noxious chemical stimuli.

4. The method of claim 1, wherein said organs are visceral organs located in the thoracic and abdominal cavity.

5. The method of claim 1, wherein said individual is diagnosed with a disease or disorder, a pathophysiological condition, or a precursor of the pathophysiological condition.

6. The method of claim 5, wherein said pathophysiological condition is an acute or chronic disease or disorder, an autoimmune disease or disorder, or a pathogen-related disease.

7. The method of claim 6, wherein the acute or chronic disease or disorder is pancreatitis.

8. The method of claim 1, wherein the drug comprises:
resiniferatoxin or derivatives thereof, including analogs and homologs of said drug such as capsaicin.

9. The method of claim 1, wherein the application of the drug to one organ comprises:
application of said drug, by means of instillation or other routes of application.

10. A method of preventing or treating a pathophysiological condition in an individual, comprising:
applying a drug to one organ, wherein said drug ablates a specific nerve response in a second organ, thereby preventing or treating the pathophysiological condition in the individual.

11. The method of claim 10, wherein the drug comprises:
resiniferatoxin or derivatives thereof, which include the analogs and homologs of said drug, such as capsaicin.

12. The method of claim 10, wherein the application of the drug to one organ comprises application of said drug, by means of instillation or other routes of application.

13. The method of claim 10, wherein said pathophysiological condition is an acute or chronic disease or disorder, an autoimmune disease or disorder, or a pathogen-related disease.

14. A method of preventing or treating a pathophysiological condition in an individual, comprising:
applying a drug to an organ, wherein said drug increases gastric emptying and reduces nausea in the individual, thereby preventing or treating the pathophysiological condition in the individual.

15. The method of claim 14, wherein the drug comprises:
resiniferatoxin or derivatives thereof including analogs and homologs of said drug.

16. The method of claim 14, wherein the application of the drug to one organ comprises:
application of said drug, by means of instillation or other routes of application.

17. The method of claim 14, wherein said pathophysiological condition is an acute or chronic disease or disorder, an autoimmune disease or disorder, or a pathogen-related disease.

18. The method of claim 17, wherein the acute or chronic disease or disorder is gastroparesis.
